(19) United States
(12) Patent Application Publication
(10) Pub. No.: US 2006/0269040 A1
(43) Pub. Date: Nov. 30, 2006

(54) Mammography method and apparatus for forming a tomosynthetic 3-D X-ray image

(76) Inventor: Thomas Mertelmeier, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/436,372

(22) Filed: May 17, 2006

(30) Foreign Application Priority Data
May 17, 2005 (DE) 10 2005 022 543.8

(51) Int. Cl. A61B 6/04

(52) U.S. Cl. 378/37

(57) ABSTRACT

In a mammography method and a mammography device recorded at different projection angles and a series of second digital individual images recorded at different projection angles are combined into at least one tomosynthetic 3D X-ray image composed of a series of layer images.

### BACKGROUND OF THE INVENTION

1. Field of the Invention

The present invention relates to a mammography method of the type wherein digital individual images recorded in a series at different projection angles are combined into a tomosynthetic 3D X-ray image. Furthermore, the invention relates to a mammography device suitable for performing such a method.

2. Description of the Prior Art

Mammography is an X-ray examination of the female breast for the purpose of detecting tumors at the earliest possible stage. Continual improvements in the mammography method have as their goal the production of more descriptive X-ray images in order to distinguish between benign and malignant changes and to reduce the number of erroneous findings, that is, the number of suspect findings resulting from non-malignant changes, and the number of malignant tumors not discovered. In conventional X-ray mammography, a two-dimensional image of the compressed breast is produced in a single projection direction (direction of incident radiation). Since in such a projection the tissue layers lying behind one another in the direction of the X-ray beam are superimposed, strongly absorbing benign structures can overlay a malignant tumor and hinder the detectability thereof.

In order to avoid this problem, for instance, a mammography method known as tomosynthesis is described in T. Wu et al., Tomographic mammography using a limited number of low-dose cone-beam projection images, Med. Phys. 30, 365 (2003), in which individual images are recorded of the female breast from a series of different projection directions of projects with a digital X-ray detector. From these digital individual images recorded from different projection angles, that is, from the image data belonging to these individual image, a series of layer images can then be constructed that model the layers of the breast oriented in parallel to the receiving surface of the X-ray detector. Such an image data set obtained from reconstruction will be called a tomosynthetic 3D X-ray image below. Due to this procedure, deeper tissue structures in the direction of propagation of the X-ray beam can be better detected.

For the improvement of tumor detection, moreover, a digital mammography method is known from Roberta A. Jong et al., “Contrast-enhanced Digital Mammography: Initial Clinical Experience,” Radiology 2003, Vol. 228, pp. 842-850, in which, after recording a first two-dimensional individual image, the patient is injected intravenously with a contrast agent that propagates in the blood vessels of the breast. From a series of individual images recorded in the same projection direction after the injection, the temporal propagation of the contrast agent can be visualized, this propagation being richer at malignant lesions. The kinematics, that is, the temporal profile of the enrichment, also indicate the presence of a malignant tumor.

From J. M. Lewin et al., “Dual-energy Contrast-enhanced Digital Subtraction Mammography: Feasibility,” Radiology 2003, Vol. 229, pp. 261-268, a further development of this contrast-enhanced digital mammography method is known in which a first individual image is recorded with a low-energy X-ray beam and a second individual image is recorded with a high-energy X-ray beam. The energy spectrum of the low-energy X-ray beam is selected such that the contrast agent is practically invisible in the first individual image, while the higher-energy X-ray beam is strongly absorbed by the contrast agent. From these individual images, a reference image is generated in which the structures of the normal breast tissue are largely eliminated and in which the contrast agent, which may propagate only weakly due to the strong compression of the breast, is more clearly visible.

### SUMMARY OF THE INVENTION

An object of the present invention is to provide a mammography method and a mammography device that allow X-ray images to be generated with increased descriptiveness relative to the state of the art.

This object is achieved in accordance with the invention by a mammography method wherein a series of first digital individual images is recorded at different projection angles and a series of second digital individual images is recorded at different projection angles, and the first and second digital individual images are combined to form at least one tomosynthetic 3D X-ray composed of a series of layer images.

By this method, tomosynthetic 3D images of the breast can be produced that have an increased descriptiveness in comparison with traditional tomosynthetic 3D X-ray images.

As used herein, the term “tomosynthetic 3D X-ray image” designates an image data set representing a series of flat, two-dimensional layer images.

In an embodiment, the first and second individual images are generated using respective X-ray beams that differ in energy, making it possible to generate a tomosynthetic 3D X-ray image from which healthy tissue disturbing the detectability of tumors or micro-calcifications is largely eliminated. The X-ray beams of different energy can be generated from different operating parameters of the X-ray tubes used to generate the individual images, for instance the tube voltage or anode filter or a combination thereof. In this case, the first and second individual images are recorded sequentially in time. This can proceed, for instance, such that for each projection angle a first individual image is recorded and then a second image is recorded after changing the operating parameters of the X-ray tube. It is basically also possible to adjust successive different projection angles with a constant adjustment of the operating parameters and to record the first individual images, and then afterwards to record the second individual images with different projection angles. The project angles at which these first individual images are recorded need not necessarily correspond to the projection angles at which the second individual images are recorded. For instance, a first individual image may be recorded at a first adjusted projection angle, a second individual image with a second adjusted projection angle, and then a first individual image again at a third adjusted projection angle, so that upon each change in position of the X-ray tube, a first individual image and second individual image are obtained successively.

In an embodiment, an energy-discriminating X-ray detector is used and the detector signals it detects are evaluated in different energy windows, so for each projection angle the first and second individual images are generated simultaneously using a broad-spectrum X-ray beam. This technique can be used to double the number of individual images used for tomosynthesis without additional time being required.

In a preferred embodiment of the invention, the tomosynthetic 3D X-ray image obtained from the first and second individual images is a tomosynthetic 3D difference image. Such a subtraction method can be used to eliminate structures in a particularly simple manner that hinders tumor detection. The tomosynthetic 3D difference image can be formed by a difference image being generated from each pair of first and second individual images recorded at the same projection angle with X-ray beams of different energy, and the multiple difference images obtained in this manner are then combined into the tomosynthetic 3D difference image.

As an alternative, the first individual images can be combined into a first or standard tomosynthetic 3D image and the second individual images can be combined into a second standard tomosynthetic image, and the tomosynthetic 3D difference image is then generated from the first and second standard tomosynthetic 3D images.

In another embodiment of the invention, after generation of a first tomosynthetic 3D X-ray image, a contrast agent is injected intravenously, and then at least one, but preferably a series, of additional tomosynthetic 3D X-ray images is generated. This measure allows malignant tumor tissue to be shown more clearly in the 3D X-ray image.

After injection of the contrast agent, a series of tomosynthetic 3D X-ray images are generated, allowing the kinetics of the contrast agent propagation is be tracked and used as an additional indicator of the existence of a malignant tumor tissue.

The above object also is achieved in accordance with the present invention by a mammography apparatus operating according to the method described above. The mammography apparatus has advantages corresponding to the advantages discussed above in connection with the method.

The above object also is achieved in accordance with the present invention by a computer-readable medium encoded with a computer program that, when loaded into a control unit or control computer of a mammography apparatus, causes the mammography apparatus to be operated in accordance with the method described above.

### DESCRIPTION OF THE DRAWINGS

FIG. 1 schematically illustrates a mammography device according to the invention.

FIGS. 2 through 4 are flowcharts in which method steps according to the invention are shown.

### DESCRIPTION OF THE PREFERRED EMBODIMENTS

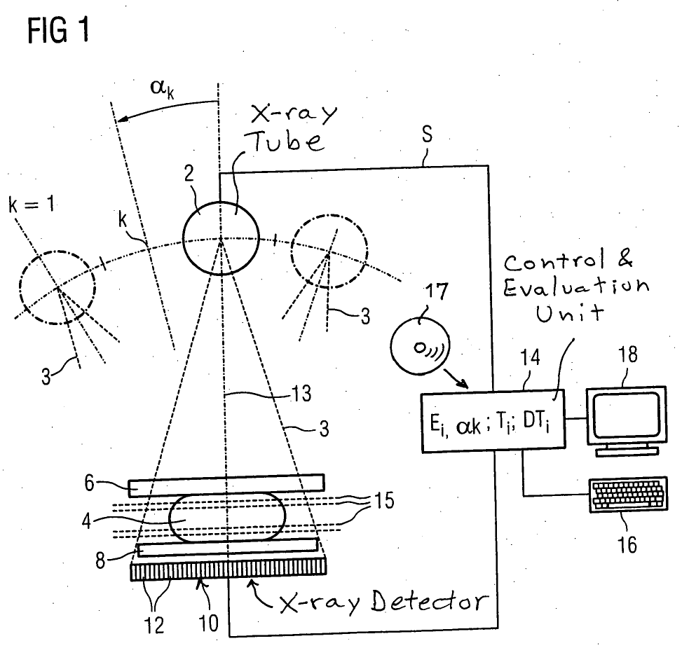

As shown in FIG. 1, the mammography device has an X-ray tube 2 for the generation of X-rays 3 that penetrate an examination object 4. The examination object 4 is a female breast that is held between a compression plate 6 and a support plate 8. The X-rays 3 penetrating the examination object 4, the compression plate 6, and the support plate 8 are received by a large-surface, particularly an energy-discriminating, digital X-ray detector 10, which is constructed of a number of individual detectors 12 arranged in a matrix-like array. The detector 10 has a detection surface 11 that is parallel to the compression plate 6 and the support plate 8.

The X-ray tube 2 may be pivoted together with the X-ray detector 10 through different angular positions k=1 . . . n, so that individual images E_{i,d,k} can be recorded of the examination object 4 at different projection angles αₖ relative to the normal 13 of the detection surface 11 of the X-ray detector 10. These individual images E_{i,d,k} are combined by reconstruction in a control and evaluation system 14 contained in an image computer into a tomosynthetic 3D X-ray image Tᵢ,DTᵢ, which is composed of a series of layer images that show different object layers 15 parallel to the support plate 8.

Control of angular position k of the X-ray tube 2 and its operating parameters is performed by control signal S that is generated by the control and evaluation system 14. Using input and display elements, shown symbolically in the example by a keyboard 16 and a monitor 18, different method variants explained in the following can be selected and executed by the user.

The control and evaluation system 14 can be loaded with any suitable computer-readable medium such as, for example, a CD ROM 17, encoded with a computer program for causing the control and evaluation unit 14 to operate the mammography apparatus shown in FIG. 1 according to one or more embodiments of the method described below.

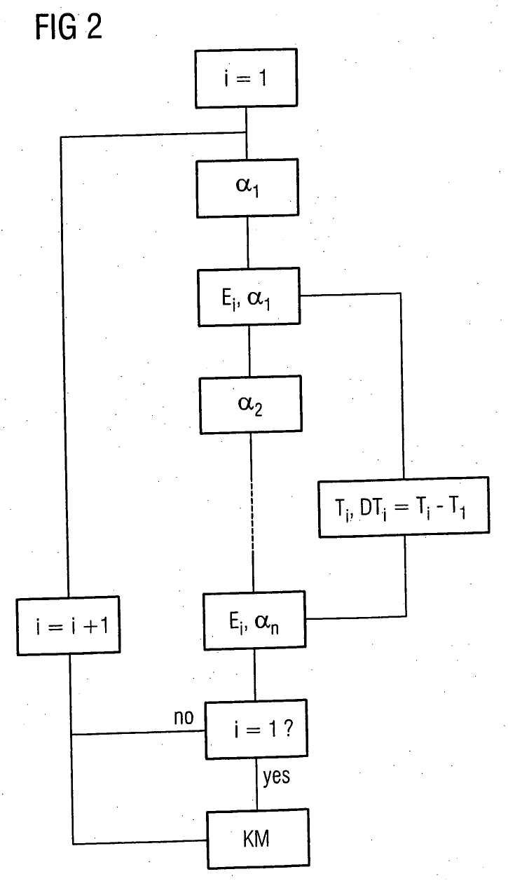

In the flowchart in FIG. 2, a method variant is shown in which a series of tomosynthetic 3D

X-ray images Tᵢ, DTᵢ is generated. To do this, at fixed operating parameters of the X-ray source, in a first step (i=1), for a series of angular positions k. (projection angles αₖ, k=1 . . . n), digital individual images E_{i,ak} (E_{1,a1} . . . E_{1,an}) are generated, which are combined into a tomosynthetic 3D X-ray image, which is a standard tomosynthetic 3D X-ray image (hereinafter called a standard tomosynthetic 3D image Tᵢ). From the second step on (i>1), from the standard tomosynthetic 3D images Tᵢ, tomosynthetic 3D X-ray images are generated through calculation of differences, which are hereinafter called 3D difference images DTᵢ=Tᵢ−Tᵢ.

After generation of a first standard tomosynthetic 3D image Ti a contrast agent KM is introduced, and digital individual images E_{i,a1} through E_{i,an} are again recorded in different angular positions k (projection angles α₁ through αₙ) and combined into standard tomosynthetic 3D images Tᵢ and tomosynthetic 3D difference images DTᵢ. Using the tomosynthetic difference images DTᵢ, the kinetics of the contrast agent propagation can be visualized and analyzed.

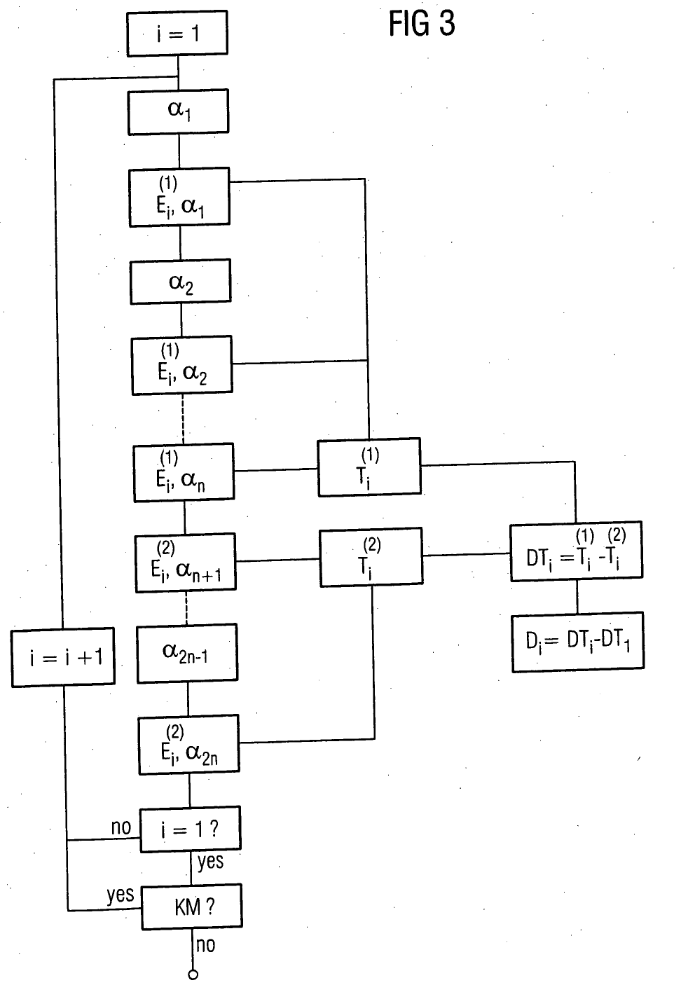

In the alternative variant according to FIG. 3, in different angular positions k=1 through k=n (projection angles α₁ through αₙ), individual images E_{1,ak} are generated from X-ray beams having a first energy spectrum. The individual images are combined into a standard tomosynthetic 3D image T⁽¹⁾i. Then a scan is performed with X-ray beams having a different energy spectrum, again at different angular positions k=n+1 through k=2n (projection angles αₙ₊₁ through α₂ₙ). Each projection angle αₙ₊₁ through α₂ₙ of angular positions k=n+1 through k=2n can correspond to a projection angle αₙ through an of angular positions k=1 through k=n. Thus a first series of individual image recordings can be obtained, for example, by rotating the X-ray tube 8 from a starting position k=1 into a final position k=n. By means of a following reversal of the rotational motion, a second series of individual recordings at the same projection angles (αₙ₊₁=αₙ and α₂ₙ=α₁) is produced. From these second individual images E⁽²⁾_{1,an+1} through E⁽²)_{1,a2n}, a standard tomosynthetic 3D image T⁽²⁾i is generated. From the first standard tomosynthetic 3D image T⁽¹⁾i and the second standard tomosynthetic 3D image T⁽²⁾i, a 3D difference image DTᵢ=T⁽¹⁾ᵢ−T⁽²)ᵢ is generated. After the generation of the first tomosynthetic 3D difference image DTᵢ it is asked whether the examination subject should be injected with contrast agent KM. If “yes,” the method is repeated at least once after injection of the contrast agent and at least one additional tomosynthetic 3D difference image DTᵢ (i>1) is generated, and another difference image Dᵢ=DTᵢ−DT₁ is formed.

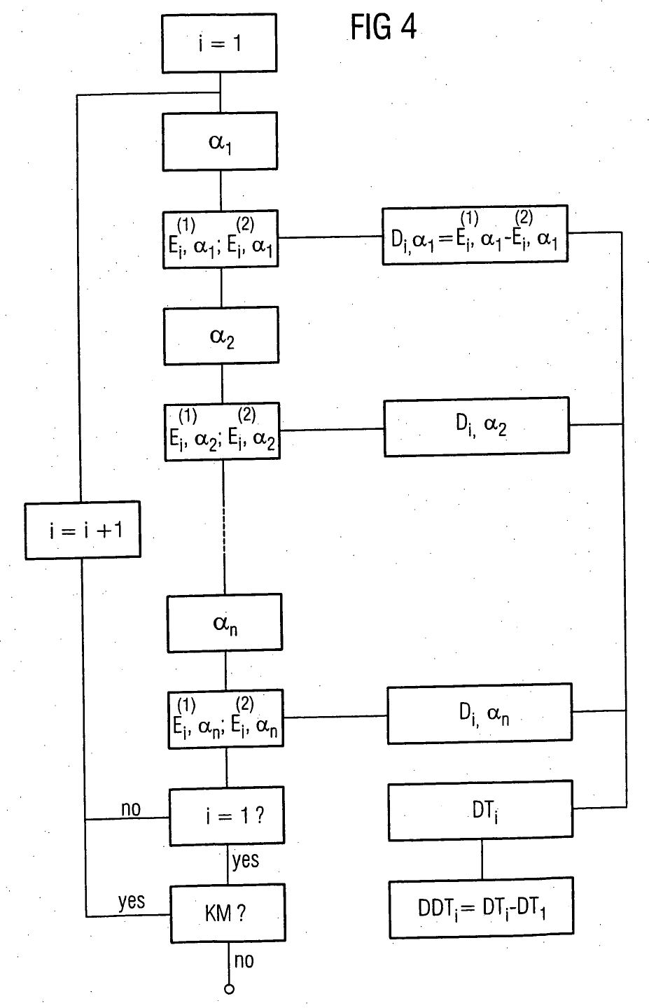

In the method flow shown in FIG. 4, in each angular position k (α₁ through αₙ), a series of individual images E⁽¹⁾_{1,ak} and E⁽²⁾_{1,ak} are generated with low-energy and high-energy X-ray beams, respectively. This can either occur simultaneously in an energy-discriminating detector, or in the same angular position αₖ at successive points in time with the X-ray tube 10 operating with different operating parameters. From these individual images E⁽¹⁾_{1,ak} and E⁽²⁾_{1,ak} recorded in each angular position k (α₁ through αₙ), difference images D_{1,ak}=E⁽¹⁾_{1,ak}−E⁽²⁾_{1,ak} are generated and combined into a tomosynthetic 3D difference image DTᵢ. In this method variant, as well, a query occurs whether a contrast agent KM should be injected. If “yes,” the same method is repeated until a given number, so that from the tomosynthetic 3D difference images DTᵢ, 3D difference images DDTᵢ can be produced.

Although modifications and changes may be suggested by those skilled in the art, it is the intention of the inventor to embody within the patent warranted hereon all changes and modifications as reasonably and properly come within the scope of his contribution to the art.

I claim as my invention:

1. A mammography method comprising the steps of:
irradiating a breast with X-rays from a plurality of different projection angles to obtain a series of first digital individual images respectively for said different projection angles;
irradiating the breast with X-rays from a plurality of different projection angles to obtain a series of second digital individual images for said different projection angles; and
electronically combining said first digital individual images and said second digital individual images to form at least tomosynthetic 3D X-ray image comprised of a series of layer images.

2. A mammography method as claimed in claim 1 comprising irradiating the breast with X-rays at a first energy when obtaining said series of first digital individual images and irradiating the breast with X-rays at a second energy, differing from said first energy, when obtaining said series of second digital individual images.

3. A mammography method as claimed in claim 2 comprising obtaining said series of said first digital individual images at respective points in time that differ from respective points in time at which said series of said second digital individual images is obtained.

4. A mammography method as claimed in claim 3 comprising obtaining said series of second digital individual images only after all of said series of first digital individual images is obtained.

5. A mammography method as claimed in claim 1 wherein the respective projection angles at which said series of first digital individual images is obtained are the same as the projection angles at which the series of second digital individual images is obtained and comprising, for each of said projection angles, simultaneously generating one of said first digital individual images and one of said second individual images by irradiating the breast with a broad-spectrum X-ray beam and detecting X-rays attenuated by the breast using an energy-discriminating X-ray detector that produces a detector signal, and evaluating said detector in respectively different energy windows so that said series of first digital individual images is obtained at a first energy of said broad-spectrum X-ray beam and said series of second digital individual images is obtained at a second energy, differing from said first energy, of said broad-spectrum X-ray beam.

6. A mammography method as claimed in claim 5 comprising obtaining said series of said first digital individual images at respective points in time that differ from respective points in time at which said series of said second digital individual images is obtained.

7. A mammography method as claimed in claim 6 comprising obtaining said series of second digital individual images only after all of said series of first digital individual images is obtained.

8. A mammography method as claimed in claim 1 comprising generating said tomosynthetic 3D X-ray image as a tomosynthetic 3D difference image by subtracting said series of first digital individual images and said series of second digital individual images from each other.

9. A mammography method as claimed in claim 8 wherein the projection angles at which the first digital individual images are respectively acquired are the same as the projection angles at which the second digital individual images are respectively acquired, and comprising acquiring said series of first digital individual images based on a first X-ray beam energy and acquiring said series of second digital individual images based on a second energy, differing from said first energy, of said X-ray beam.

10. A mammography method as claimed in claim 8 comprising forming said difference image by combining said series of first digital individual images into a first standard tomosynthetic 3D image, and combining said series of second digital individual images into a second standard tomosynthetic 3D image, and subtracting said first standard tomosynthetic 3D image and said second standard tomosynthetic 3D image from each other.

11. A mammography method as claimed in claim 1 wherein said tomosynthetic 3D X-ray image is a first tomosynthetic 3D image, and wherein the breast is the breast of a patient, and comprising the additional steps of:
after generating said first tomosynthetic 3D image, intravenously injecting the subject with a contrast agent; and
obtaining another series of first digital individual images of the breast at different projection angles and obtaining another series of second digital individual images at different projection angles, and combining said another series of first digital individual images and said another series of second digital individual images into at least one additional tomosynthetic 3D X-ray image.

12. A mammography method as claimed in claim 11 comprising:
after injecting said contrast agent, acquiring a plurality of series of first digital individual images at different projection angles and acquiring a plurality of series of second digital individual images at different projection angles, and combining respective ones of said plurality of series of first digital individual images with respective ones of said plurality of series of second digital individual images respectively into a plurality of tomosynthetic 3D X-ray images.

13. A mammography method as claimed in claim 12 comprising generating a plurality of difference tomosynthetic 3D X-ray images by subtracting said first tomosynthetic 3D X-ray image from each of said plurality of tomosynthetic 3D X-ray images to visualize kinetics of said contrast agent in the breast.

14. A mammography apparatus comprising the steps of:
a movable X-ray source and a radiation detector operable to irradiate a breast with X-rays from a plurality of different projection angles to obtain a series of first digital individual images respectively for said different projection angles with said radiation detector, and to irradiate the breast with X-rays from a plurality of different projection angles to obtain a series of second digital individual images for said different projection angles with said radiation detector; and
a computer that electronically combines said first digital individual images and said second digital individual images to form at least tomosynthetic 3D X-ray image comprised of a series of layer images.

15. A computer-readable medium encoded with a computer program and being loadable into a control and evaluation unit of a mammography apparatus for causing said control and evaluation unit to operate the mammography apparatus to:
irradiate a breast with X-rays from an X-ray source at a plurality of different projection angles to obtain a series of first digital individual images respectively for said different projection angles with a radiation receiver;
irradiate the breast with X-rays from the X-ray source at a plurality of different projection angles to obtain a series of second digital individual images for said different projection angles with the radiation receiver; and
in said control and evaluation unit, electronically combine said first digital individual images and said second digital individual images to form at least tomosynthetic 3D X-ray image comprised of a series of layer images.
